(19) United States
(12) Patent Application Publication
(10) Pub. No.: US 2005/0287553 A1
(43) Pub. Date: Dec. 29, 2005

(54) Method for the quantification of methylated DNA

(75) Inventors: David Guetig, Berlin (DE); Dirk Habighorst, Berlin (DE); Antje Kluth, Berlin (DE); Armin Schmitt, Berlin (DE); Matthias Schuster, Berlin (DE); Ina Schwope, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 11/100,779

(22) Filed: Apr. 6, 2005

(30) Foreign Application Priority Data
Apr. 6, 2004 (EP) 04 090 133.2
May 28, 2004 (EP) 04 090 213.2

(51) Int. Cl. C12Q 1/68; C12P 19/34

(52) U.S. Cl. 435/6; 435/91.2

(57) ABSTRACT

Particular aspects of the present invention provide a method for quantification of two different variations of a DNA sequence. Particularly, the invention relates to a quantification of methylated DNA, and for this purpose, the test DNA is converted so that cytosine is converted to uracil, while 5-methylcytosine remains unchanged. The converted DNA is amplified by means of a real-time PCR, wherein two labeled real-time probe types are utilized: one specific for methylated DNA; and one for unmethylated DNA. Preferably, the degree of methylation of the test DNA is calculated from the ratio of the signal intensities of the probes or from the Ct values. The inventive methods have substantial utility for diagnosis and prognosis of cancer and other disorders associated with altered or characteristic DNA methylation status, as well as having substantial utility for analysis of SNPs, allelic expression, and prediction of drug response, drug interactions, among other uses.

### CROSS-REFERENCE TO RELATED APPLICATIONS

This application claims the benefit of priority to European Patent Applications EP 04 090 133.2, filed 06 Apr. 2004, entitled “Verfahren zur Quantifizierung methylierter DNA,” and EP 04 090 213.2, filed 28 May 2004, of same title, both of which are incorporated by reference herein in their entirety.

### FIELD OF THE INVENTION

Aspects of the present invention relate generally to DNA methylation, and more particularly to novel compositions and methods for the quantification of methylated cytosine positions in DNA, and for quantification of allelic expression, and sequence and strain variations.

### BACKGROUND

The base 5-methylcytosine is the most frequent covalently modified base found in the DNA of eukaryotic cells. DNA methylation plays an important biological role in, for example, regulating transcription, genetic imprinting, and tumorigenesis (for review see, e.g., Millar et al.: Five not four: History and significance of the fifth base; in The Epigenome, S. Beck and A. Olek (eds.), Wiley-VCH Publishers, Weinheim 2003, pp. 3-20). Identification of 5-methylcytosine is of particular interest in the area of cancer diagnosis. Cytosine and 5-methylcytosine have the same base-pairing behavior, making 5-methylcytosine difficult to detect using particular standard methods. The conventional DNA analysis methods based on hybridization, for example, are not applicable.

Accordingly, current methods for DNA methylation analysis are based on two different approaches. The first approach utilizes methylation-specific restriction enzymes to distinguish methylated DNA, based on methylation-specific DNA cleavage. The second approach comprises selective chemical conversion (see, e.g., bisulfite treatment; see e.g., PCT/EP2004/011715) of unmethylated cytosines (but not methylated cytosines) to uracil. The enzymatically or chemically pretreated DNA generated in these approaches is typically amplified and analyzed in different ways (see, e.g., WO 02/072880 pp. 1 ff; Fraga and Estella: DNA methylation: a profile of methods and applications; Biotechniques, 33:632, 634, 636-49, 2002). Chemically pretreated DNA is generally amplified by means of a PCR method, providing good sensitivity. Additionally, selective amplification only of methylated (or with the reverse approach, unmethylated) DNA is attained by using methylation-specific primers in so-called methylation-sensitive PCR (MSP) methods, or by using ‘blockers’ in “Heavy Methy™” methods (see, e.g., Herman et al.: Methylation-specific PCR: a novel PCR assay for methylation status of CpG islands. Proc Natl Acad Sci USA. 93:9821-6, 1996; Cottrell et al.: A real-time PCR assay for DNA-methylation using methylation-specific blockers. Nucl. Acids Res., 32:e10, 2004). Alternatively, it is possible to amplify the DNA in a non-methylation-specific manner, and analyze the amplificates by means of methylation-specific probes (see, e.g., Trinh et al.: DNA methylation analysis by MethyLight technology. Methods, 25:456-62, 2001). Particular PCR-based methods are also applicable as ‘real-time’ PCR variants, making it possible to detect methylation status directly in the course of the PCR, without the need for a subsequent analysis of the products (MethyLight™; WO 00/70090; U.S. Pat. No. 6,331,393; and Trinh et al. 2001, supra).

Quantification of the degree of DNA methylation. Quantification of the degree of DNA methylation is required in many assays including, but not limited to, classification of tumors, obtaining prognostic information, or for predicting drug effects/responses, and different methods of such quantification are known in the art, such as ‘end-point analysis’ and ‘threshold-value analysis.’

End-point analyses. Amplification of the DNA is produced, in part, for example, with Ms-SNuPE, with hybridizations on microarrays, with hybridization assays in solution or with direct bisulfite sequencing (see, e.g., Fraga and Estella 2002, supra). A problem with such “end point analyses” (where the amplificate quantity is determined at the end of the amplification) is that the amplification can occur non-uniformly because of, inter alia, obstruction of product, enzyme instability and/or a decrease in concentration of the reaction components. Correlation between the quantity of amplificate, and the quantity of DNA utilized is, therefore, not always suitable, and quantification is thus sensitive to error (see, e.g., Kains: The PCR plateau phase—towards an understanding of its limitations. Biochem. Biophys. Acta 1494:23-27, 2000).

Threshold-value analyses. By contrast, threshold-value analysis, which is based on a real-time PCR, determines the quantity of amplificate in the exponential phase of the amplification, rather than at the end of the amplification. Such threshold, real-time methods presume that the amplification efficiency is constant in the exponential phase. The art-recognized threshold value ‘Ct’ is a measure corresponding, within a PCR reaction, to the first PCR cycle in which the signal in the exponential phase of the amplification is greater than the background signal. Absolute quantification is then determined by means of a comparison of the Ct value of the investigated (test) DNA with the Ct value of a standard (see, e.g., Trinh et al. 2001, supra; Lehmann et al.: Quantitative assessment of promoter hypermethylation during breast cancer development. Am J Pathol., 160:605-12, 2002). A substantial problem of such Ct value-based analyses is that when high DNA concentrations are used, only a small resolution can be achieved. This problem also applies when high degrees of methylation are determined via PMR values (for discussion of PMR values see, e.g., Eads et al., CANCER RESEARCH 61:3410-3418, 2001.) Additionally, amplification of a reference gene (e.g., the β-actin gene) is also required for this type of Ct analysis (see, e.g., Trinh et al. 2001, supra).

Therefore, there is a pronounced need in the art for novel and effective quantitative methods of methylation analysis. There is a pronounced need in the art for quantitative real-time methods that increase resolution over a broader range of DNA concentrations (e.g., when relatively high DNA concentration are used), and/or when high degrees of methylation are determined using PMR values. There is a pronounced need in the art for quantitative real-time methylation methods that do not require determining the absolute DNA quantity (e.g., amplification of a reference gene). There is a pronounced need in the art for rapid and reliable measurement of the relative quantity of alleles (e.g., methylated alleles), and for improved handling of diagnostic analyses (e.g., diagnostic methylation analysis).

### SUMMARY OF THE INVENTION

Particular aspects of the present invention provide a novel real-time PCR method for quantitative methylation analysis, the method comprising producing a non-methylation-specific, conversion-specific amplification of the target DNA. Amplificates are detected by means of the hybridization thereto of two different methylation-specific real-time PCR probes: one specific for the methylated state; and the other specific for the unmethylated state. Preferably, the two probes are distinguishable, for example, by bearing different labels (e.g., different fluorescent dyes). A quantification of the degree of methylation is produced within specific PCR cycles employing the ratio of signal intensities of the two probes. Alternatively, the Ct values of the two respective detection channels (e.g., fluorescent channels) can also be utilized for the methylation quantification. In both cases, a quantification of the degree of methylation is possible without the necessity of determining the absolute DNA quantity. A simultaneous amplification of a reference gene or a determination of the PMR values is thus not necessary. Significantly, the method according to the invention supplies reliable values for both large and small DNA quantities, as well as for high and low degrees of methylation.

### BRIEF DESCRIPTION OF THE DRAWINGS

FIG. 1 shows elements of a representative QM assay according to aspects of the present invention. Primers are used for the amplification, and are bisulfite-specific, but contain no CpG positions (shown as black circles). Probes, by contrast, are specific for the corresponding methylated or the unmethylated state of the respective ‘covered’ CpG positions. When both probes are used in the same reaction, they are labeled with different fluorescent dyes (R1, R2; Q=quencher).

FIGS. 2A and 2B show particular results, as disclosed in EXAMPLE 1 herein, relating to detection of amplification products of TFF1. The number of cycles of the amplification assay is displayed along the x-axis, whereas the fluorescent signal (intensity) of the hybridization probes is displayed along the y-axis. FIG. 2A shows the amplification curves of DNA mixtures of known methylation levels detected with the FAM-labeled probe for the methylated state, whereas FIG. 2B shows corresponding detection with the VIC-labeled probe for the unmethylated state.

FIGS. 3A, 3B, 3C and 3D show particular results, as disclosed in EXAMPLE 1 herein, relating to calibration curves based on fluorescent intensities in the optimal cycle (maximum of the first derivative of the amplification curve) and corresponding curve parameters. FIGS. 3A and 3B: Cycle 36 of the amplification of TFF1, 1 ng of initial DNA; FIG. 3A: slope, R², y-axis intercept; FIG. 3B: whisker plots of Fisher scores. FIGS. 3C and 3D: Cycle 35 of the amplification of S100A2, 1 ng of initial DNA; FIG. 3C: slope, R², y-axis intercept; FIG. 3D: whisker plots of Fisher scores.

FIGS. 4A and 4B show particular results, as disclosed in EXAMPLE 1 herein, relating to detection of amplification products of TFF1. FIGS. 4A and 4B: calibration curves based on Ct values and corresponding curve parameters, amplification of TFF1 on 1 ng of DNA; FIG. 4A: slope, R², y-axis intercept; FIG. 4B: whisker plots of Fisher scores.

FIGS. 5A and 5B show particular results, as disclosed in EXAMPLE 1 herein, comparing the curve parameters (slope, R², y-axis intercept, Fisher scores for differentiating adjacent methylation levels) of the calibration curves, which are obtained in different techniques for evaluation (based on fluorescent intensities in the optimal cycle or at the end point or based on Ct values) of amplification curves; FIG. 5A: amplification of S100A2 on 10 ng of initial DNA; FIG. 5B: amplification of TFF1 on 10 ng of initial DNA. The y-axis shows the values of the different quality parameters which are presented along the x-axis: a=linearity, b=slope, c=y-intercept, d=Fischer 0:5; e=Fischer 5:10; f=Fischer 10:25; g=Fischer 25:50; h=Fischer 50:75; I=Fischer 75:100. The black columns represent the present invention calculating the methylation rate by the optimal amplification cycle. The white columns represent determination by end point analysis, and the grey coulmns represent the Ct-value analysis.

FIG. 6 shows particular results as disclosed in EXAMPLE 3 herein. Methylation rate, in percent, is shown along the y-axis. Nine different samples, each of four different input bisulfite DNA amounts, were investigated: 50 ng (left bar in each group); 10 ng (second from left); 5ng (second from right); and 1 ng (right). The standard deviation does not exceed 5% in any case.

FIG. 7 shows particular results as disclosed in EXAMPLE 4 herein. Twelve (12) different QM assays were conducted in five separate runs. The methylation rate, in percent, is shown along the y-axis. The different runs showed a low intra- and inter-plate variability.

FIG. 8 shows particular results as disclosed in EXAMPLE 4 herein. Twelve (12) different QM assays were conducted in five separate runs. The methylation rate, in percent, is shown along the y-axis, whereas the x-axis displays the number of repetitions. The calculated confidence interval is about ±5 percentage points of the mean of the methylation rate.

FIG. 9 shows the results of the present EXAMPLE 5 (chip assay). The X axis shows the metastasis free survival times of the patients in years, and the Y axis shows the proportion of recurrence free survival patients in %. The lower curve shows the proportion of metastasis free patients in the population with above median methylation levels, and the upper curve shows the proportion of metastasis free patients in the population with below median methylation levels.

FIG. 10 shows the results of the present EXAMPLE 5 (QM assay). The X axis shows the metastasis free survival times of the patients in years, and the Y axis shows the proportion of recurrence free survival patients in %. The lower curve shows the proportion of metastasis free patients in the population with above median methylation levels, and the upper curve shows the proportion of metastasis free patients in the population with below median methylation levels.

FIG. 11 shows the correlation of measured methylation values using the chip platform (Y-axis) and the exemplary assay of the present invention (Y-axis) of each patient. The correlation co-efficient is 0.87.

### DETAILED DESCRIPTION OF THE INVENTION

Particular aspects of the present invention represent important technical advances by provide novel quantitative real-time methylation assay methods that provided resolution over a broad range of DNA concentrations (e.g., when relatively high DNA concentration are used), and/or when methylation (e.g., high degrees thereof) is determined using PMR values. The inventive methods do not require determining the absolute quantity of DNA (e.g., amplification of a reference gene).

More particularly, aspects of the present invention provide a novel real-time PCR method for quantitative methylation analysis, comprising producing a non-methylation-specific, conversion-specific target DNA amplification. Amplificates are detected by means of the hybridization thereto of two different methylation-specific real-time PCR probes: one specific for the methylated state; and the other specific for the unmethylated state. Preferably, the two probes are distinguishable, for example, by bearing different labels (e.g., different fluorescent dyes). A quantification of the degree of methylation is produced within specific PCR cycles employing the ratio of signal intensities of the two probes. Alternatively, the Ct values of the two respective detection channels (e.g., fluorescent channels) can also be utilized for the methylation quantification. In both cases, a quantification of the degree of methylation is possible without the necessity of determining the absolute DNA quantity. A simultaneous amplification of a reference gene or a determination of the PMR values is thus not necessary. Significantly, the method according to the invention supplies reliable values for both large and small DNA quantities, as well as for high and low degrees of methylation.

Quantitative Methylation (“QM”) Assay Embodiments

In specific aspects, the invention provides a method for the quantification of methylated DNA comprising:
a) the DNA to be investigated is reacted in such a way that 5-methylcytosine remains unchanged, while unmethylated cytosine is converted into uracil or into another base which is distinguished from cytosine in its base-pairing behavior;
b) the converted DNA is amplified in the presence of two real-time probes, wherein one of the probes is specific for the methylated state, and the other probe is specific for the unmethylated state of the DNA;
c) it is determined at different time points how far the amplification has proceeded by detecting the hybridization of the probes to the amplificates; and
d) the degree of methylation of the investigated DNA is determined.

In the first step of this exemplary QM embodiment, the DNA to be investigated (e.g., test DNA) is reacted/treated with a chemical, or with an enzyme, in such a way that 5-methylcytosine remains unchanged, whereas unmethylated cytosine is converted into uracil or into another base which is distinguishable from cytosine by virtue of its base-pairing behavior. The DNA to be investigated can originate from different sources (e.g., tissue samples, cell, cell lines, biopsies, histological slides, body fluids, or tissue embedded in paraffin), depending, for example, on the diagnostic, scientific or other applicable objective. For diagnostic objectives, tissue samples are preferably used as the initial material, but body fluids (e.g., sputum, stool, urine, or cerebrospinal fluid, ejaculate, blood plasma, blood serum, whole blood, isolated blood cells and cells isolated from the blood), particularly serum, can also be used. Preferably, the DNA is first isolated from the biological sample. Extraction may be by means that are standard to one skilled in the art, including but not limited to the use of detergent lysates, sonification and vortexing with glass beads. In particular embodiments, the DNA is extracted according to standard methods from blood, e.g., with the use of the Qiagen UltraSens DNA extraction kit. In particular embodiments, the isolated DNA is fragmented (e.g., by reaction with restriction enzymes). The reaction conditions and the enzymes employed for such isolation and fragmentation/restriction are known to a person of ordinary skill in the relevant art (e.g., from the protocols supplied by the manufacturers), and could be optimized thereby for such uses. The DNA is converted chemically or by means of enzymes. Preferably, chemical conversion by means of a reagent comprising bisulfite (a reagent comprising bisulfite, disulfite, hydrogen sulfite or combinations thereof, useful as disclosed herein to distinguish between methylated and unmethylated CpG dinucleotide sequences) is conducted. Variations of bisulfite conversion are known to persons of ordinary skill in the relevant art (see, e.g., Frommer et al.: A genomic sequencing protocol that yields a positive display of 5-methylcytosine residues in individual DNA strands. Proc Natl Acad Sci USA., 89:1827-31, 1992 (incorporated by reference herein in its entirety); Olek, A modified and improved method for bisulphite based cytosine methylation analysis. Nucleic Acids Res. 24:5064-6, 1996 (incorporated by reference herein in its entirety); and see PCT/EP2004/011715 (incorporated by reference herein in its entirety)). It is particularly preferred that the bisulfite conversion is conducted in the presence of denaturing solvents (e.g., dioxane) and a radical trap (see: PCT/EP2004/011715; incorporated by reference herein in its entirety). In another embodiment, the DNA is not chemically converted, but rather is converted by enzymes. This is possible, for example, by the use of cytidine deaminases, which convert unmethylated cytidine more rapidly than methylated cytidine. An appropriate exemplary enzyme has been identified (Bransteitter et al.: Activation-induced cytidine deaminase deaminates deoxycytidine on single-stranded DNA but requires the action of RNase. Proc Natl. Acad Sci USA. 100:4102-7, 2003).

In the second step of this exemplary QM embodiment, the converted DNA is amplified in the presence of two real-time probes, wherein one of the probes is specific for the methylated DNA state (e.g., of a test DNA CpG dinucleotide sequence), and the other probe is specific for the unmethylated DNA state. Preferably, an amplification is conducted by means of an exponential amplification process, such as PCR. Primers used for the amplification are specific for the chemically or enzymatically converted DNA. Preferably, non-methylation-specific primers are utilized (i.e., primers that encompass (do not make available) CG or methylation-specific TG or CA dinucleotide sequences/positions, providing for uniform amplification of methylated and unmethylated DNA. Alternatively, it is possible to amplify a larger sequence region in a methylation-specific manner and thus to quantify specific cytosine positions within this sequence by means of the inventive methods. The design of methylation-specific and non-methylation-specific primers, and the PCR reaction conditions are known in the art (see e.g., U.S. Pat. No. 6,331,393; Trinh et al., 2001, supra). Preferably, the primers are located close to the probe(s). Preferably, the length of the amplicon should not exceed about 200 bp. Preferably, the amplicon melting temperature, Tm, should be from about 52 to about 60° C. (e.g., depending on probe-Tm, approx. 5-7° C. below the probe-Tm).

In preferred embodiments, the amplification is conducted in the presence of two different probes, wherein one of the probes is specific for the methylated state of the target DNA, while the other probe is specific for the unmethylated state of the target DNA. The methylation-specific probes correspondingly bear (encompass) at least one CpG dinucleotide sequence/position, while the non-methylation-specific probes make available (encompass) at least one specific TG or CA dinucleotide sequence/position. Preferably, the probes bear three specific dinucleotide sequences/positions. Preferably, both probes cover the same dinucleotide positions (e.g., the same CpG-positions). Preferably, melting temperatures of the probes are similar. Preferably, the probes cover positions representing converted C-positions to ensure conversion-specific detection. Preferably, the probes comprise real-time probes (e.g., TaqMan™, etc). Such real-time probes are understood herein to be probes that permit the amplificates to be detected during the amplification process, as opposed to after. Different real-time PCR variants are familiar to persons skilled in the art, and include but are not limited to Lightcycler™, TaqMan™, Sunrise™, Molecular Beacon™ or Eclipse™ probes. The particulars on constructing and detecting these probes are known in the art (see, e.g., U.S. Pat. No. 6,331,393 with additional citations, incorporated by reference herein). The design of the probes is carried out manually, or by means of suitable software (e.g., the “PrimerExpress™” software of Applied Biosystems (for TaqMan™ probes) or via the MGB Eclipse™ design software of Epoch Biosciences (for Eclipse™ probes). Preferably, the real-time probes are selected from the probe group consisting of FRET probes, dual-label probe comprising a fluorescence-reporter moiety and fluorescence-quencher moiety, Lightcycler™, TaqMan™, Sunrise™, Molecular Beacon™, Eclipse™, scorpion-type primers that comprise a probe that hybridizes to a target site within the scorpion primer extension product, and combinations thereof. Preferably, TaqMan™ probes are used, and are utilized most preferably in combination with Minor Groove Binders (MGB).

Preferably, TaqMan™ probe design follows the Applied Biosystems design guidelines for the “TaqMan™ Allelic Discriminiation” assay, and both probes have the same 5′-end, which influences the 5′-exonuclease activity of the polymerase. Runs of identical nucleotides (e.g., >4 bases, especially G) are preferably avoided. Preferably, in fluorescence based embodiments, there is no G at the probe 5′-end (G tends to quenche the reporter fluorescence). Preferred embodiments comprise probe sequences containing more Cs than Gs, and the polymorphic site is preferably located approximately in the middle third of the sequence. Preferred reporter dyes are FAM (carboxyfluorescein) and VIC.

Amplification reactions can be conducted in one or more tubes. Preferably, the amplification is conducted together with both probes in one vessel, so that the reaction conditions for both probes are identical. This embodiment also leads to an increased specificity, because the probes compete for binding sites. The two probes bear distinguishable or different labels. Preferably, the two probes bear different labels. Alternately, the amplifications are conducted in different vessels, and in this way, disruptive interactions between the fluorescent dyes can be avoided. When performing amplifications and detection with 2 probes in 2 vessels, a competing unlabeled oligonucleotide can be used to increase the specificity of probe binding.

The third step of this exemplary QM embodiment comprises determination of the extent that amplification at different time points (i.e., determination of how far the amplification has proceeded). Determination of the extent of amplification is accomplished by detecting hybridizations during the individual amplification cycles, using art-recognized methods corresponding to, and depending on the probes utilized.

In the fourth step of this exemplary QM embodiment, the degree of methylation of the investigated DNA (test DNA) is determined, by using one of various means, including but not limited to means based on: the fluorescent signal intensities; the first derivative of the fluorescent intensity curves; or the ratio of threshold values at which a certain signal intensity will be exceeded (e.g., at the ‘Ct’ values).

In a preferred embodiment, the degree of methylation of the investigated DNA is determined from the ratio of the signal intensities of the two probes. Preferably, such determination is by means of the following formula:
M=100*I_{CG}/(I_{CG}+I_{TG}),
where the notation “I_{CG}” indicates the signal intensity of the probe specific for the methylated DNA state, and “I_{TG}” indicates the signal intensity of the probe specific for the unmethylated DNA state. Determining the signal intensity ratios during a PCR cycle in the exponential amplification phase of the PCR is particularly preferred. Preferably such calculation is carried out close to (or at) the cycle in which the amplification reaches its maximal increase, corresponding to the point of inflection of the fluorescent intensity curve or the maximum of its first derivative. The calculation is thus conducted at a time point which preferably lies at up to five cycles before or after the inflection point, particularly preferably up to two cycles before or after the inflection point, and most particularly preferred up to one cycle before or after the inflection point. In the optimal embodiment, the calculation occurs directly at the inflection point. In cases where the inflection points of the two curves (corresponding to the two probes) lie in different cycles, the calculation is preferably conducted at the inflection point of the curve which has the highest signal at this time point.

Alternatively, determination of the inflection point is made by means of the first derivative of the fluorescent intensity curves. The first derivatives are preferably first subjected to a smoothing “Spline” (see, e.g., Press, W. H., Teukolsky, S. A., Vetterling, W. T., Flannery, B. P. (2002). Numerical Recipes in C. Cambridge: University Press; Chapter 3.3).

In yet another embodiment, the calculation of the degree of methylation is conducted by means of the ratio of threshold values at which a certain signal intensity will be exceeded (e.g., at the ‘Ct’ values, rather than by means of the ratio of the fluorescent intensities. Determination of Ct values is known in the art (see, e.g., Trinh et al., 2002, supra). The degree of methylation can then be determined via the following formula:
degree of methylation=100/(1+2^{ΔCt}).

In yet further embodiments, other criteria for calculating the degree of methylation are used (e.g., the area under the fluorescent curve (area under the curve), the maximal slope of the curves, or the maximum of the second derivative of amplification).

In particular embodiments, quantification of the degree of methylation is facilitated and optimized by use of standards (standard samples). Specifically, such optimization is conducted using different DNA methylation standards; for example, corresponding to 0%, 5%, 10%, 25%, 50%, 75% and 100% degree of DNA methylation. Preferably, DNA that covers the entire genomic DNA is used. Alternately, a representative portion of such DNA is used as the standard. Standard samples haveing different degrees of methylation are obtained by appropriate mixtures of methylated and unmethylated DNA. The production of methylated DNA is relatively simple with the use of Sss1 methylase, which converts all unmethylated cytosines in the sequence context CG to 5-methylcytosine. Sperm DNA, which provides only a small degree of methylation, can be used as completely unmethylated DNA (see, e.g., Trinh et al., 2001, supra.).

The preparation of unmethylated DNA is preferably conducted by means of a so-called ‘genome-wide’ amplification (WGA—whole genome amplification; see, e.g., Hawkins et al.: Whole genome amplification—applications and advances. Curr Opin Biotechnol., 13:65-7, 2002). With WGA, wide parts of the genome will be amplified by means of “random” or degenerate primers. A completely unmethylated DNA results after several amplification cycles, because only unmethylated cytosine nucleotides will be provided in the amplification. Preferably, a “Multiple Displacement Amplification” (MDA) is produced by means of φ29 polymerase (see, e.g., Dean et al., 2002, supra; and U.S. Pat. No. 6,124,120). Similarly produced DNA is available from different commercial suppliers (e.g., “GenomiPhi” of Amersham Biosciences; “Repli-g” of Molecular Staging).

The production of methylation standards is described in great detail, for example, in European Patent Application 04 090 037.5, filed: 05 Feb. 2004; applicant: Epigenomics AG). The measured ‘methylation rate’ is obtained by calculating the quotient of the signals which are detected for the methylated state, and the sum of the signals which are detected for the methylated and the unmethylated state. A ‘calibration curve’ is obtained if this quotient is plotted against the theoretical methylation rates (corresponding to the proportion of methylated DNA in the defined mixtures), and the regression line that passes through the measured points is determined. A calibration is conducted preferably with different quantities of DNA; for example, with 0.1, 1 and 10 ng of DNA per batch.

Assays are particularly suitable for quantification according to the invention, where the calibration curves for the time point of the exponential amplification provide a y-axis crossing as close as possible to zero. Methylation states that are adjacent should be distinguished by a high Fisher score (preferably greater than 1, and more preferably greater than 3). Additionally, it is advantageous if a y-axis intercept is provided that is as small as possible, and a Fisher score is provided that is as high as possible (preferably greater than 1, and more preferably greater than 3). Preferably, the curves have a slope and a regression close to the value 1. The assays can be optimized in these respects by means of varying the primers, the probes, the temperature program, and the other reaction parameters using standard tests, as will be appreciated by those of skill in the art.

While, as described above, the ‘methylation rate’ can be determined with the inventive methods independently from a standard curve, the ‘absolute content of methylated DNA’ can be readily determined by using the inventive methods in conjunction with a standard curve as described herein.

Diagnosis and/or Prognosis of Cancer and other Disorders or Conditions Characterized by Altered or Characteristic Methylation Status

A particularly preferred use of the inventive methods lies in the diagnosis and/or prognosis of cancer diseases, or other disorders or conditions associated with a change of DNA methylation status. These include, but are not limited to: CNS malfunctions; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction.

In alternate embodiments, the inventive methods have substantial utility for predicting subject/drug or subject/treatment interactions (e.g., drug responsiveness, or undesired interactions, etc.), for the differentiation of cell types or tissues, or for the investigation of cell differentiation.

Kits

Methylation kits are also provided by aspects of the present invention, where such kits comprise two primers, a polymerase, a probe specific for the methylated state, and a probe specific for the unmethylated state, and, optionally, additional reagents necessary for a PCR, and/or a bisulfite reagent.

Determination of Sequence Differences, and of Strain Diffences

As will be appreciated by those of skill in the relevant art, the above-described inventive embodiments can be used not only for the methylation analysis, but also for the quantification of sequence differences in RNA or in DNA. For these applications, the first step of the described method—the chemical or enzymatic conversion—is not conducted. Thus, it is possible to investigate allele-specific gene expression, or a gene duplication by means of the inventive methods. Additionally, it is possible to investigate single nucleotide polymorphisms (SNPs) from pooled samples. Another application of the inventive methods is quantification of different strains of microorganisms.

Therefore, the present invention provides a method for quantification of two different variations of a DNA sequence, comprising:
a) the DNA is amplified in the presence of two real-time probes, wherein one of the probes is specific for one variation of the DNA sequence, and the other probe is specific for the other variation of the DNA sequence;
b) it is determined at different time points how far the amplification has proceeded by detecting the hybridization of the probes to the amplificates; and
c) the proportions of the two sequence variations is determined.

According to further aspects of the present invention, all of the above-described inventive embodiments (e.g., methods, uses, kits, etc.) are applicable to (can be applied in the context of) outside the sphere of methylation analysis (e.g., are applicable to diagnosis, prognosis, determination of sequence differences, determination of strain differences, etc.), and the resulting applications are thus also encompassed within the scope of the present invention. Such modifications and variations will be recognized by those of skill in the art, based on the present enabling disclosure and teachings.

Therefore, additional embodiments provide a kit comprising two primers, a polymerase, a probe specific for one variation of the DNA sequence, and a probe specific for the other variation of the DNA sequence to be investigated. The kit may optionally contain additional reagents necessary for a PCR

Allele-Specific Gene Expression

Additional embodiments of the present invention provide a method for the investigation of allele-specific gene expression (for review see, e.g., Lo et al.: Allelic variation in gene expression is common in the human genome. Genome Res., 13:1855-62, 2003; Weber et al.: A real-time polymerase chain reaction assay for quantification of allele ratios and correction of amplification bias. Anal Biochem 320:252-8, 2003). These inventive applications comprise an initial reverse transcription of the RNA to be analyzed. Particular specific embodiments provide a method for the quantification of allele-specific gene expression, comprising:
a) the RNA to be investigated is reverse-transcribed;
b) the cDNA is amplified in the presence of two real-rime probes, whereby one of the probes is specific for one of the alleles and the other probe is specific for the other allele;
c) at different time points it is determined how far the amplification has proceeded by detecting the hybridizations of the probes to the amplificates; and
d) the allele-specific gene expression is quantified.

In the first step of such embodiments, the RNA to be investigated is reverse-transcribed. Appropriate methods are found in the prior art (see, e.g., Lo et al. 2003, supra; incorporated by reference herein in its entirety). Typically, the RNA is isolated first. Various commercially available kits can be used for this purpose (e.g., Micro-Fast Track, Invitrogen; RNAzol™ B, Tel-Test™). The cDNA is then produced by means of a commercially available reverse transcriptase (e.g., such as that from Invitrogen).

In the second step of this exemplary embodiment, the cDNA is amplified in the presence of two real-time probes, wherein one of the probes is specific for the sequence of one allele, and the other probe is specific for the sequence of the other allele. The probes correspond to real-time probes or FRET-based probes (e.g., Lightcycle™, Taqman™, Sunrise™, Molecular Beacon or Eclipse™ probes). Details relating to constructing and detecting these probes are well known in the prior art as discussed herein above.

Preferably, the amplification is conducted by means of an exponential amplification process, and most preferably by means of a PCR. Primers are used for the amplification, and such primers preferably amplify the DNA of both alleles in a uniform manner. The design of appropriate primers and probes, as well as the PCR reaction conditions, are familiar to those of skill in the relevant art (see above). Preferably, the amplification is conducted together with both probes in one amplification reaction vessel, so that the reaction conditions for both probes are identical (see above).

In the third step of such inventive embodiments, the extent of amplification is determined at different time points (i.e., determination of how far the amplification has proceeded). This is done, for example, by detecting the hybridizations of the probes to the amplificates (e.g., by means of labels attached to the probes) during the individual amplification cycles. Suitable probe detection methods are known in the art, and depend on the particular probes utilized (see above).

In the fourth step of such inventive embodiments, the allele-specific gene expression is quantified. As described herein above (in relation to methylation analysis), this can be achieved in various ways. In a preferred embodiment, quantification is made by means of the ratio of signal intensities of the two probes. However, it is also possible to utilize the area under the corresponding fluorescent curves or the maximal slope of the curves for quantifying the ratio of the threshold values (see above).

As was described in detail for the methylation analysis embodiments, quantification is is additionally facilitated if the assay conditions have been previously optimized in these respects. For this purpose, a calibration curve is plotted by means of a standard series which contains different proportions of the two allele sequences of interest. The quality criteria (e.g., y-axis intercept, Fisher score, slope regression) described in detail for the methylation analysis are also generally applicable to the instant embodiments.

Single Nucleotide Polymorphisms (SNPs) Analyses

Yet further embodiments of the present invention, while distinguishable from those of the above-described methylation analysis, provide methods for investigation of single nucleotide polymorphisms (SNPs) from pooled samples. A pool of samples is meaningful for different objectives, such as for identifying genes which take part in the emergence of complex disorders (see, e.g., Shifman et al.: Quantitative technologies for allele frequency estimation of SNPs in DNA pools. Mol Cell Probes 16:429-34, 2002). Therefore, specific embodiments provide a method for investigating SNPs from pooled samples, comprising:
a) the sample to be investigated is amplified in the presence of two real-time probes, whereby one of the probes is specific for the sequence of one SNP, and the other probe is specific for the sequence of the other SNP;
b) at different time points it is deterimed how far the amplification has proceeded by detecting the hybridizations of the probed to the amplificates; and
c) it is concluded from this which SNP at what fraction is represented in the pool.
   A gene duplication event can also be investigated according to the these principles (see also, e.g., Pielberg et al.: A sensitive method for detecting variation in copy numbers of duplicated genes. Genome Res 13:2171-7, 2003).
   Investigation of Strain Differences or Mutations in Microorganisms

Additional aspects of the present invention provide methods for investigation of strain differences and/or mutations in microorganisms. According to such inventive embodiments, the proportion of wild type and the proportion of mutant strain (or the relative proportions of two different strains) is determined in a sample. Such applications can be of significant importance for therapeutic decisions (see, e.g.: Nelson et al.: Detection of all single-base mismatches in solution by chemiluminescence. Nucleic Acids Res 24:4998-5003, 1996). A specific embodiment provides a method for determining the proportion of wild type and mutant strains in a mixed sample, comprising:
a) the sample to be investigated is amplified in the presence of two real-time probes, whereby one of the probes is specific for the sequence of the wild type, and the other probe is specific for the sequence of the mutant strain;
b) at different time points it is determined how far the amplification has proceeded (e.g., by detecting the hybridizations of the probes to the amplificates); and
c) from this, the fractional representation of the strain in the sample is determined.

### EXAMPLE 1

### The Degree of Methylation of the Two Genes S100A2 and TFF1 was Analyzed

Particular aspects of the present invention provide for a reliable quantification of DNA methylation. For this purpose, the degree of methylation of the two genes S100A2 and TFF1 will be analyzed.

Calibration curves with several DNA mixtures of different degrees of methylation were plotted. A series of DNA mixtures of known degrees of methylation were used as the standard (0, 5, 10, 25, 50, 75 and 100% methylated DNA). For the production of this “gold standard,” completely methylated and completely unmethylated DNA were mixed together in different ratios. The completely unmethylated DNA was obtained from Molecular Staging, where it was prepared by means of a multiple displacement amplification of human genomic DNA from whole blood. The completely methylated DNA was produced by means of an Sss1 treatment of the completely unmethylated DNA according to the manufacturer's instructions. The DNA was then bisulfite-converted (see PCT/EP2004/011715; incorporated by reference herein in its entirety).

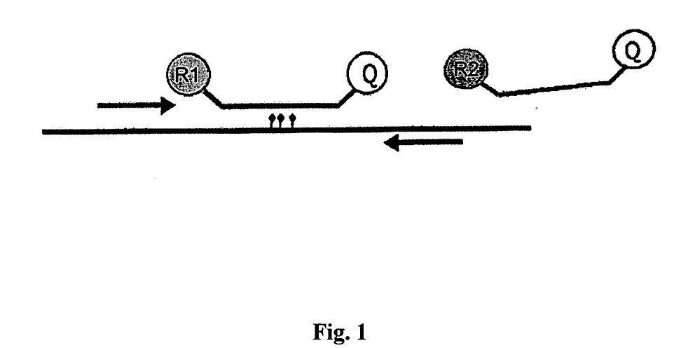

For the real-time PCR assays, primer pairs were used which were specific for the bisulfite conversion. The primers, however, were nonspecific for methylation (i.e., they did not contain CpG positions). Two bisulfite-specific MGB-Taqman probes (Applied Biosystems) were also utilized. These probes comprised 2 CpG positions. One probe was specific for the methylated state and was labeled with FAM. The second probe was specific for the unmethylated state and bore a VIC label (see FIG. 1).

The following primers and probes were used for TFF1: methylation-specific probe, 6-FAM-ACA CCG TTC GTa aaa-MGBNFQ (SEQ ID NO:1); non-methylation-specific probe, VIC-ACA CCA TTC Ata aaa T-MGBNFQ (SEQ ID NO:2); Forward Primer, AGt TGG TGA TGt TGA TtA GAG tt (SEQ ID NO:3); Reverse Primer, and CCC TCC CAa TaT aCA AAT AAa aaC Ta (SEQ ID NO:4).

The following oligonucleotides were utilized for S100A2: methylation-specific probe, 6-FAM-tTC GTG Tat ATA tAT GCG ttT G-MGBNFQ (SEQ ID NO:5); non-methylation-specific probe, VIC-tTT GTG Tat ATA tAT GTG ttT GTG-MGBNFQ (SEQ ID NO:6); Forward Primer, Ttt TGT GTG AGA GGt TGT GAG tAt (SEQ ID NO:7); and Reverse Primer, CCT CCT aAT aTC CCC CAa CT (SEQ ID NO:8).

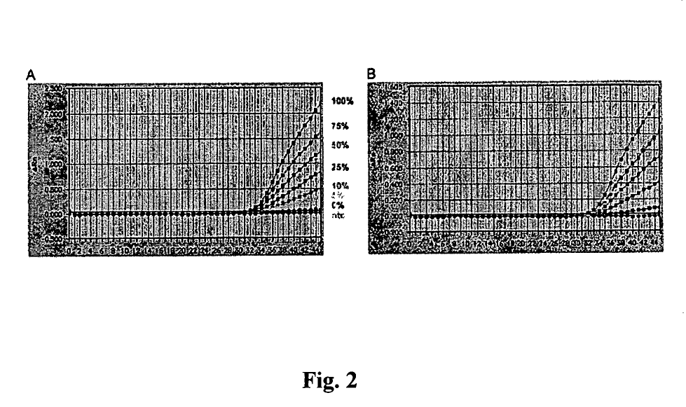

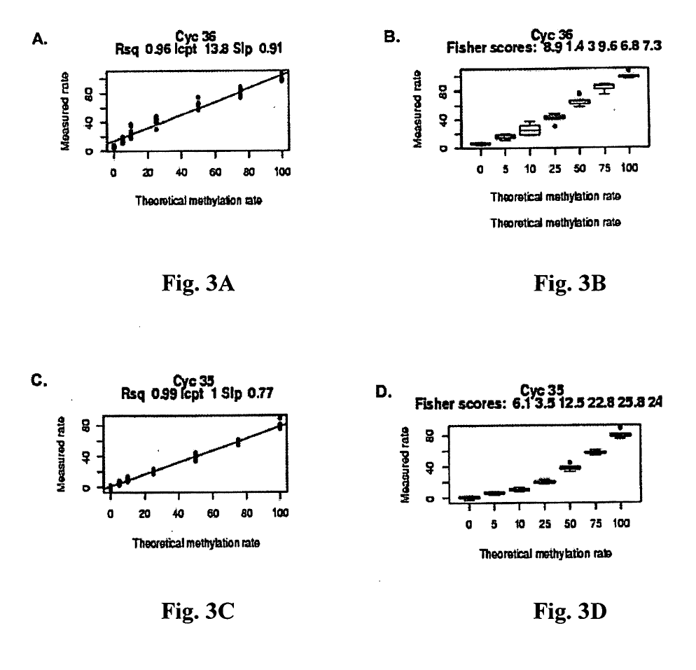

The real-time PCR was carried out in an ABI7700 Sequence Detection System (Applied Biosystems) in a 20 μl reaction volume. The final concentrations in the reaction mixtures amounted to: 1×TaqMan Buffer A (Applied Biosystems) containing ROX as a passive reference dye, 2.5 mmol/l MgCl₂ (Applied Biosystems), 1 U of AmpliTaq Gold DNA polymerase (Applied Biosystems), 625 nmol/l primers, 200 nmol/l probes, and 200 μmol/l dNTPs. The temperature profile for the TFF1 assay was conducted as follows: 10 min activation at 94° C., followed by 45 cycles of 15 s at 94° C. denaturing and 60 s at 60° C. annealing+elongation. The fluorescence was measured during the 60° C. step (FIG. 2). The annealing was conducted at 62° C. for the S100A2 assay. The data analysis was conducted according to the recommendations of Applied Biosystems. The degrees of methylation were determined according to the following formula: methylation rate:=delta Rn CG probe/(delta Rn CG probe+delta Rn TG probe). By plotting the measured methylation rates against the theoretical methylation rates, a calibration curve was prepared for each PCR cycle (FIG. 3). The suitability of the individual curves for the quantification was determined by means of the following curve parameters: slope; R²; y-axis intercept; as well as Fisher scores for the classification of adjacent methylation levels each time (FIG. 3).

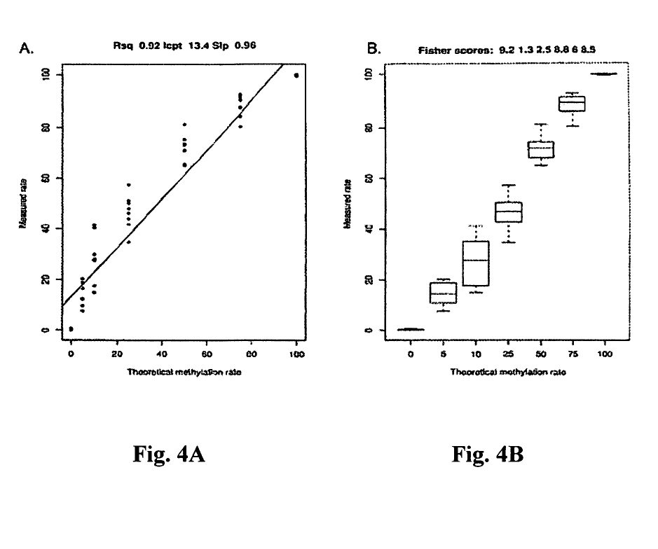

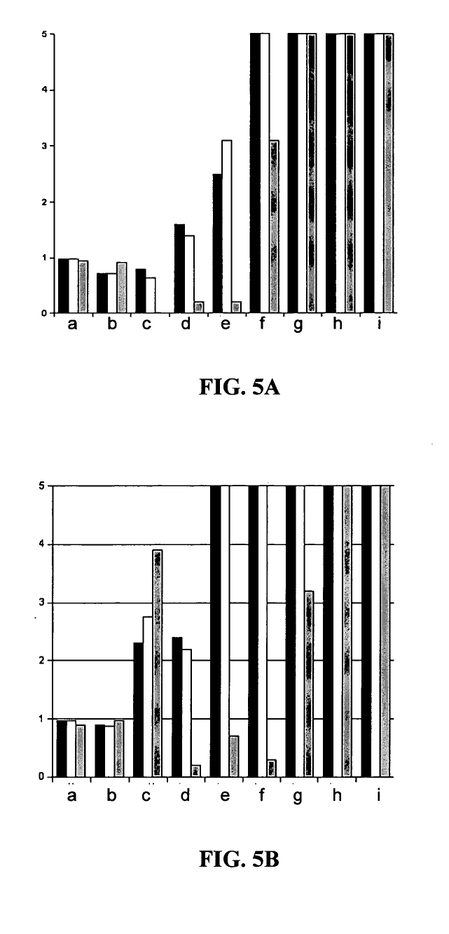

From the same experiments, calibration curves were plotted on the threshold cycles (Ct), wherein the methylation rate was calculated with the following formula: methylation rate=100/(1+2^{delta Ct} (FIG. 4). If the suitability of the different cycles (optimal cycle, in which the slope of the amplification curve is maximal, vs. final cycle) is compared with the suitability of the calibration based on Ct values, it can be seen that overall the calibration by means of the optimal cycle produces the best curve parameters (FIG. 5); namely, slope close to 1, R² close to 1, y-axis intercept close to 0, and Fisher scores>1.

### EXAMPLE 2

### Inventive Methods Were Used to Provide a Reliable Quantification of the Methylation of Different Types of Samples

Additional aspects of the present invention provide for a reliable quantification of the methylation of different types of samples. For this purpose, a portion of the biological sample material was fresh frozen, and the remainder was embedded in paraffin. Using standard, art-recognized techniques, the DNA was then isolated from the sample, and was treated with a bisulfite reagent (see, e.g. PCT/EP2004/011715, incorporated by reference herein in its entirety). The treated DNA was amplified by means of two non-methylation-specific primers in the presence of two Taqman oligonucleotide probes. One of the oligonucleotide probes was specific for the methylated state, and the other for the unmethylated state of the investigated gene. Both probes had a reporter fluorescent dye at the 5′-end and a quencher at the 3′-end. The reactions were calibrated with DNA standards of a defined methylation status as described above.

The β-actin gene (ACTB) was used/investigated for determining the quantity of sample DNA. The primers and probes utilized here did not provide CpG dinucleotides, so that the amplification was produced here independently of the methylation status. Thus only one probe was necessary here. The following oligonucleotides were used: Primer 1, TGG TGA TGG AGG AGG TTT AGT AAG T (SEQ ID NO:9); Primer 2, AAC CAA TAA AAC CTA CTC CTC CCT TAA (SEQ ID NO:10); and probe, 6-FAM-ACC ACC ACC CAA CAC ACA ATA ACA AAC ACA-TAMRA or Dabcyl (SEQ ID NO.11).

The following reaction componenents were utilized: 3 mmol/l MgCl₂ buffer; 10×buffer; and Hotstart TAQ. The following temperature program was used: 95° C. for 10 minutes; then 45 cycles: 95° C.; 15 sec; and 62° C., 1 min. The fluorescent signals were recorded with a Lightcycler™ device. The degree of methylation of a specific locus was determined by the following formula: degree of methylation=100 * I_{CG}/(I_{CG}+I_{TG}); where I=fluorescent intensity of the CG or TG probe.

Table 1 shows the results of this EXAMPLE 2. “Fresh” denotes fresh frozen tissue, “PET” stands for paraffin-embedded tissue. In all, 18 sample pairs were investigated, and it was shown that the inventive method allows for quantification from both types of samples.

TABLE 1
Investigation, according to particular aspects
of the present invention, of 18 sample pairs.

| SAMPLE | Methylation Rate |
|---|---|
| Fresh 1 | 56.72 |
| PET1 | 51.99 |
| Fresh 2 | 4.74 |
| PET 2 | 11.13 |
| Fresh 3 | 8.56 |
| PET 3 | 12.22 |
| Fresh 4 | 52.3 |
| PET 4 | 58.67 |
| Fresh 5 | 54.51 |
| PET 5 | 62.91 |
| Fresh 6 | 27.76 |
| PET 6 | 39.24 |
| Fresh 7 | 6.18 |
| PET 7 | 2.48 |
| Fresh 8 | 15.06 |
| PET 8 | 7.18 |
| Fresh 9 | 9.97 |
| PET 9 | 12.18 |
| Fresh 10 | 59.52 |
| PET 10 | 72.26 |
| Fresh 11 | 22.29 |
| PET 11 | 29.62 |
| Fresh 12 | 4.39 |
| PET 12 | 7.63 |
| Fresh 13 | 19.07 |
| PET 13 | 39.62 |
| Fresh 14 | 35.13 |
| PET 14 | NA |
| Fresh 15 | 10.27 |
| PET 15 | 11.1 |
| Fresh 16 | 9.08 |
| PET 16 | 45.3 |
| Fresh 17 | 42.66 |
| PET 17 | 38.64 |
| Fresh 18 | 28.67 |
| PET 18 | 18.38 |

### EXAMPLE 3

### Reliability of the QM Assay Was Demonstrated Over a Broad Range of Input DNA

Experiments were preformed to demonstrate that the inventive QM assays perform well over a wide range of input DNA amounts. Different amounts of bisulfite-treated DNA (50, 10, 5, and 1 ng) derived from nine different samples (e.g., fresh frozen tissue samples, and paraffin embedded tissue samples) were analyzed by the inventive QM assay.

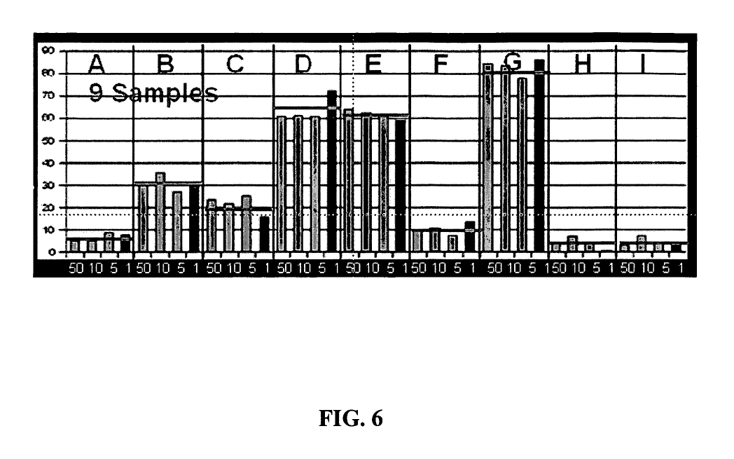

The results are illustrated in FIG. 6, which shows that the QM assays perform well over a wide range of input DNA. The determined methylation degree is independent of the DNA input amount. The standard deviation does not exceed a value of ±5 percentage points around the mean of measured methylation rate. This value of the standard deviation is caused by the interplate variablity (see Example 4 below).

### EXAMPLE 4

### Reproducibility of QM Assay was Demonstrated

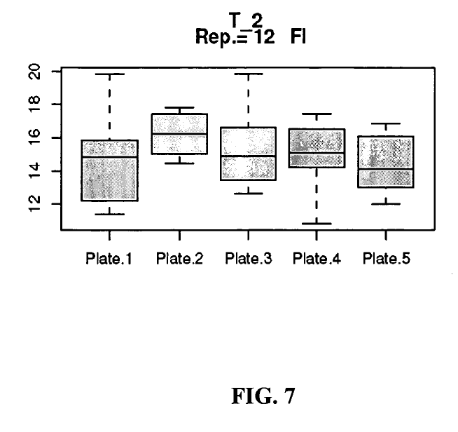

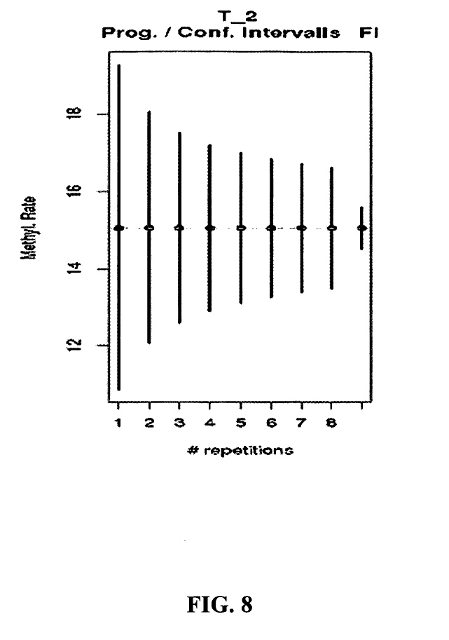

To investigate the reproducibility of the QM assay 12 different QM assays were conducted in five separate runs. As indicated in FIG. 7, the assays showed a low intra- and inter-plate variability. The confidence interval is around ±5 percentage points of the mean of the methylation rate (FIG. 8).

### EXAMPLE 5

### Methylation Analysis by Means of Array (“Chip”) Analysis was Compared to the Inventive Assays

Methylation of the gene PITX2 was analyzed in patients with breast cancer to provide a comparison of methylation analysis by means of array (“chip”) analysis to the assays of the present invention.

The following study was based on samples from 236 breast cancer patients, wherein all patients were NO (nodal status negative), and older than 35 years. In all cases surgery was performed before 1998. All patients were ER+ (estrogen receptor positive), and the tumors were graded to be Ti-3, G1-3. In this study all patients received Tamoxifen directly after surgery, and the outcome was assessed according to the length of disease-free survival.

The DNA samples were extracted using the Wizzard™ Kit (Promega). Total genomic DNA of all samples was bisulfite treated converting unmethylated cytosines to uracil, while methylated cytosines remained conserved. Bisulfite treatment was performed with minor modifications according to the protocol described in Olek et al., 1996; incorporated by reference herein). After bisulfitation, 10 ng of each DNA sample was used in subsequent mPCR reactions containing 6-8 primer pairs. Each reaction contained the following: 2.5 pmol each primer; 11.25 ng DNA (bisulfite treated); and Multiplex PCR Master mix (Qiagen). The primer oligonucleotides used to generate the amplificate, were: GTAGGGGAGGGAAGTAGATGT (SEQ ID NO: 12); TCCTCAACTCTACAAACCTAAAA (SEQ ID NO: 13). Initial denaturation was carried out at 95° C. for 15 min. Forty cycles were carried out as follows: denaturation at 95° C. for 30 sec, followed by annealing at 57° C for 90 sec.; primer elongation at 72° C. for 90 sec.; and final elongation at 72° C. was carried out for 10 min. All PCR products from each individual sample were then hybridised to glass slides carrying a pair of immobilised oligonucleotides for each CpG position under analysis. Each of these detection oligonucleotides was designed to hybridise to the bisulphite converted sequence around one CpG site which was either originally unmethylated (TG) or methylated (CG). Hybridisation conditions were selected to allow the detection of the single nucleotide differences between the TG and CO variants. Five (5) μl volume of each multiplex PCR product was diluted in 10×Ssarc buffer. The reaction mixture was then hybridised to the detection oligonucleotides as follows: denaturation at 95° C.; cooling down to 10° C.; and hybridisation at 42° C. overnight, followed by washing with 10×Ssarc and dH20 at 42° C. The sequences of the oligonucleotides used were the following: AGT CGG GAG AGC GAA A (SEQ ID NO:14); and GTT GGG AGA GTG AAA (SEQ ID NO:15).

Fluorescent signals from each hybridised oligonucleotide were detected using genepix scanner and software. Ratios for the two signals (from the CG oligonucleotide and the TG oligonucleotide used to analyse each CpG position) were calculated based on comparison of intensity of the fluorescent signals.

The log methylation ratio (log(CG/TG)) at each CpG position is determined according to a standardised pre-processing pipeline that includes the following steps: for each spot, the median background pixel intensity is subtracted from the median foreground pixel intensity (this gives a good estimate of background corrected hybridisation intensities); for both CG and TG detection oligonucleotides of each CpG position, the background corrected median of 4 redundant spot intensities is taken; for each chip and each CpG position, the log(CG/TG) ratio is calculated; and for each sample the median of log(CG/TG) intensities over the redundant chip repetitions is taken. This ratio has the property that the hybridisation noise has approximately constant variance over the full range of possible methylation rates (Huber et al., 2002).

The same samples were then analysed by means of the assay of the present invention. The amount of sample DNA amplified was quantified by reference to the gene (β-actin (ACTB)) to normalize for input DNA. For standardization the primers and the probe for analysis of the ACTB gene lacked CpG dinucleotides so that amplification is possible regardless of methylation levels. As there are no methylation variable positions, only one probe oligonucleotide is required.

The following oligonucleotides were used in the reaction to amplify the control amplificate: Control Primer1, TGG TGA TGG AGG AGG TTT AGT AAG T (SEQ ID NO:16); Control Primer2, AAC CAA TAA AAC CTA CTC CTC CCT TAA (SEQ ID NO:17); and Control Probe, 6FAM-ACC ACC ACC CAA CAC ACA ATA ACA AAC ACA-TAMRA or Dabcyl (SEQ ID NO:18).

The following primers are used to generate an amplificate within the PITX2 sequence comprising the CpG sites of interest: Primers for PITX bisulfite amplificate length (144 bp PITX2), GTA GGG GAG GGA AGT AGA TGT T (SEQ ID NO:19); and PITX2, TTC TAA TCC TCC TTT CCA CAA TAA (SEQ ID NO:20). The probes used were: PITX2cg1, FAM-AGT CGG AGT CGG GAG AGC GA-Darquencher (SEQ ID NO:21); and as an alternative quencher TAMRA was also used in additional experiments, FAM-AGT CGG AGT CGG GAG AGC GA-TAMRA; PITX2tg1: YAKIMA YELLOW-AGT TGG AGT TGG GAG AGT GAA AGG AGA-Darquencher (SEQ ID NO:22).

The extent of methylation at a specific locus was determined by the following formula: methylation rate=100 * I (CG)/(I(CG)+I(TG)); where I=Intensity of the fluorescence of CG-probe or TG-probe).

PCR components were ordered from Eurogentec: 3 mM MgCl2 buffer; 10×buffer; Hotstart TAQ; and using the following program (45 cycles): 95° C., 10 min; 95° C., 15 sec; and 62° C., 1 min.

### Results

For each assay the methylation (and where relevant mean methylation over multiple oligo-pairs) for each amplificate was calculated and the population split into groups according to their mean methylation values, wherein one group was composed of individuals with a methylation score higher than the median and a second group composed of individuals with a methylation score lower than the median.

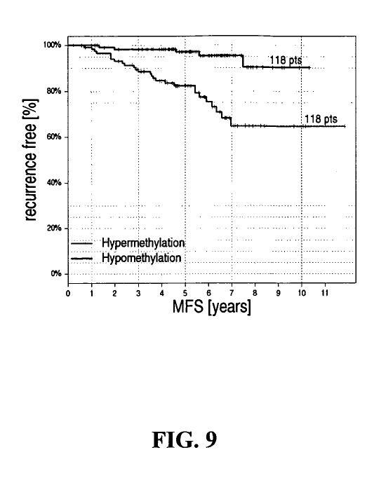

Results are shown in FIGS. 9 to 11. FIG. 9 shows the results of chip assay. The X axis shows the metastasis free survival times of the patients in years, and the Y axis shows the proportion of recurrence free survival patients in %. The lower curve shows the proportion of metastasis free patients in the population with above median methylation levels, and the upper curve shows the proportion of metastasis free patients in the population with below median methylation levels.

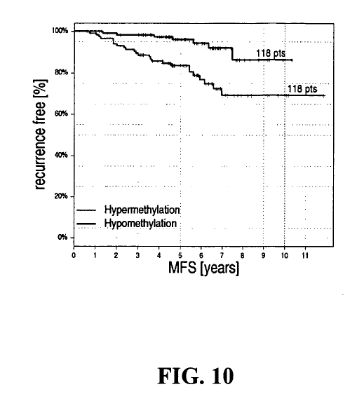

FIG. 10 shows, the results of the QM assay. The X axis shows the metastasis free survival times of the patients in years, and the Y axis shows the proportion of recurrence free survival patients in %. The lower curve shows the proportion of metastasis free patients in the population with above median methylation levels, and the upper curve shows the proportion of metastasis free patients in the population with below median methylation levels.

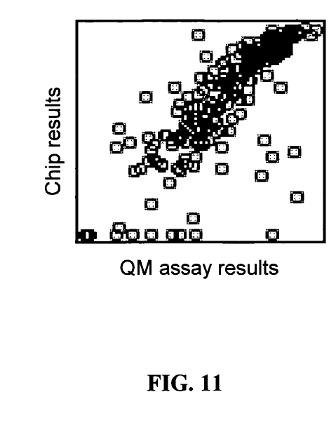

FIG. 11 shows the correlation of measured methylation values using the chip platform (Y axis) and the exemplary assay of the present invention (Y-axis) of each patient. The correlation co-efficient is 0.87.

Therefore, the survival curves generated by microarray analysis were substantially confirmed by the new QM assay (FIGS. 9 and 10). The correlation plot between microarry and QM assay is shown in FIG. 11, indicating a co-efficient of 0.87. Therefore, methylation markers pre-validated by microarray methylation analysis are well transferable to the QM-assay format.

1. A method for the quantification of methylated DNA, comprising:
a) contacting isolated DNA with a reagent or series of reagents suitable to convert unmethylated cytosine to uracil or to another base that is distinguishable from cytosine, while leaving 5-methylcytosine unchanged;
b) amplifying the converted DNA, or a portion thereof, in the presence of two real-time probes having respective detectable labels, and wherein one of the amplificate probes is specific for a methylated state of at least one CpG dinucleotide sequence of the isolated DNA, and the other probe is specific for the corresponding unmethylated state of the isolated DNA; and
c) determining, based on the detectable labels, and at one or more different time points during the amplification, the extent of amplification, whereby the degree of methylation of the investigated DNA is, at least in part, determined.

2. The method of claim 1, wherein amplifying is by means of an exponential amplification method.

3. The method of claim 2, wherein amplifying is by means of a polymerase chain reaction (PCR).

4. The method of claim 1, wherein amplifying comprises use of primers that are not methylation-specific.

5. The method of claim 1, wherein the real-time probes are selected from the probe group consisting of Lightcycler, Taqman, Sunrise, Molecular Beacon, Eclipse, and combinations thereof.

6. The method of claim 5, wherein Taqman probes are utilized in combination with minor groove binders as real-time probes.

7. The method of claim 1, wherein the respective detectable labels of the probes are distinguishable, and amplifying is conducted in the presence of both probes in a single reaction vessel.

8. The method according to claim 1, wherein the degree of methylation is determined from a ratio of the signal intensities of the two probes at a specific time point.

9. The method of claim 8, wherein amplifying is by means of an exponential amplification method, and wherein the degree of methylation is determined from a ratio of the signal intensities at a time point during the exponential amplification phase.

10. The method of claim 9, wherein the degree of methylation is determined at a time point that lies at or within about 5 cycles before or after the time point at which the amplification reaches its maximal slope, as determinable from the inflection point of corresponding fluorescent intensity curves.

11. The method of claim 10, wherein the degree of methylation is determined at a time point that lies at or within about 2 cycles before or after the time point at which the amplification reaches its maximal slope.

12. The method of claim 11, wherein the degree of methylation is determined at a time point that lies at or within 1 cycle before or after the time point at which the amplification reaches its maximal slope.

13. The method of claim 12, wherein the degree of methylation is determined at a time point at which the amplification reaches its maximal slope.

14. The method of claim 1, wherein the degree of methylation is determined by means of a ratio of threshold values at which a particular signal intensity is exceeded.

15. The method of claim 14, wherein the determination is by means of a ratio of Ct values.

16. The method of claim 15, wherein the determination is by means of the following formula: degree of methylation=100/(1+2^{ΔCt}).

17. The method of claim 1, wherein the degree of methylation is determined by means of a ratio of the area under corresponding fluorescent intensity curves, or by means of the maximal slope of the curves.

18. The method of claim 2, further comprising, prior to amplifying in b), optimizing the assay conditions: to minimize, or substantially minimize, the y-axis intercept of corresponding fluorescent intensity curves; and to maximize, or substantially maximize, a Fisher score for a time point of the exponential amplification.

19. The method claim 2, further comprising, prior to amplifying in b), optimizing the assay conditions so that corresponding fluorescent intensity curves have a slope and a regression close to the value 1 for a time point of the exponential amplification.

20. The method of claim 1, further comprising determining an absolute degree of methylation by use of a standard curve based on the proportion of methylated DNA in defined mixtures of methylated and unmethylated DNA.

21. The method of claim 1, wherein quantification of methylated DNA is carried out for the diagnosis or prognosis of cancer, or for other disorders or conditions associated with an altered or characteristic DNA methylation status.

22. The method of claim 1, wherein quantification of methylated DNA is carried out for a purpose selected from the group consisting of: predicting drug responses; predicting adverse drug interactions, differentiation of cell types or tissues, and investigation of cell differentiation.

23. A kit, comprising: two primer oligomers; a polymerase suitable for primer-based DNA amplification; a probe specific for a methylated DNA state; and a probe specific for the corresponding unmethylated DNA state.

24. The kit of claim 23, further comprising at least one of: additional PCR reagents, a bisulfite reagent; and reagents for generating a standard curve based on the proportion of methylated DNA in defined mixtures of methylated and unmethylated DNA.

25. A method for the quantification of two different variations of a DNA sequence comprising:
a) amplifying isolated DNA, or a portion thereof, in the presence of two real-time probes having respective detectable labels, and wherein one of the amplificate probes is specific for one sequence variation of the isolated DNA, and the other probe is specific for another sequence variation of the isolated DNA; and
b) determining, based on the detectable labels, and at one or more different time points during the amplification, the extent of amplification, whereby the proportions of the two sequence variations are determined.

26. A kit, comprising: two primer oligomers; a polymerase suitable for primer-based DNA amplification; a probe specific for one variation of a DNA sequence; and a probe specific for another variation of the DNA sequence.

27. The kit of claim 26, further comprising additional PCR reagents.

28. A method for quantification of allele-specific gene expression, comprising:
a) reverse transcribing RNA to generate a corresponding cDNA;
b) amplifying the cDNA, or a portion thereof, in the presence of two real-time probes having respective detectable labels, and wherein one of the amplificate probes is specific for one allele, and the other probe is specific for another allele; and
c) determining, based on the detectable labels, and at one or more different time points during the amplification, the extent of amplification, whereby allele-specific gene expression is quantified.

29. A method for the investigation of SNPs from pooled samples, comprising:
a) amplifying isolated DNA, or a portion thereof, in the presence of two real-time probes having respective detectable labels, and wherein one of the amplificate probes is specific for one SNP, and the other probe is specific for another SNP; and
b) determining, based on the detectable labels, and at one or more different time points during the amplification, the extent of amplification, whereby characteriztion of the SNPs is achieved.

30. A method for determining the relative fractions two microorganism strains in a mixed sample, comprising:
a) amplifying isolated microorganism DNA, or a portion thereof, in the presence of two real-time probes having respective detectable labels, and wherein one of the amplificate probes is specific for one strain of microorganism, and the other probe is specific for another strain; and
b) determining, based on the detectable labels, and at one or more different time points during the amplification, the extent of amplification, whereby the relative fractions of two microorganism strains is determined.

31. The method of claim 30, wherein one of the strains is a wild-type strain, and the other is a variant or mutant strain thereof.


SEQUENCE LISTING (22 sequences)

SEQ ID NO: 1 - DNA, 15 bp, Artificial sequence
  TFF1 methylation-specific probe (FAM--MGBNFQ) for chemically treated genomic DNA
acaccgttcgtaaaa

SEQ ID NO: 2 - DNA, 16 bp, Artificial sequence
  TFF1 non-methylation-specific probe (VIC--MGBNFQ) for chemically treated genomic DNA
acaccattcataaaat

SEQ ID NO: 3 - DNA, 23 bp, Artificial sequence
  TFF1 forward primer for chemically treated genomic DNA
agttggtgatgttgattagagtt

SEQ ID NO: 4 - DNA, 26 bp, Artificial sequence
  TFF1 reverse primer for chemically treated genomic DNA
ccctcccaatatacaaataaaaacta

SEQ ID NO: 5 - DNA, 22 bp, Artificial sequence
  S100A2 methylation-specific probe (FAM--MGBNFQ) for chemically treated genomic DNA
ttcgtgtatatatatgcgtttg

SEQ ID NO: 6 - DNA, 24 bp, Artificial sequence
  S100A2 non-methylation-specific probe (VIC--MGBNFQ) for chemically treated genomic DNA
tttgtgtatatatatgtgtttgtg

SEQ ID NO: 7 - DNA, 24 bp, Artificial sequence
  S100A2 forward primer for chemically treated genomic DNA
ttttgtgtgagaggttgtgagtat

SEQ ID NO: 8 - DNA, 20 bp, Artificial sequence
  S100A2 reverse primer for chemically treated genomic DNA
cctcctaatatcccccaact

SEQ ID NO: 9 - DNA, 25 bp, Artificial sequence
  Beta-actin forward primer for chemically treated genomic DNA
tggtgatggaggaggtttagtaagt

SEQ ID NO: 10 - DNA, 27 bp, Artificial sequence
  Beta-actin reverse primer for chemically treated genomic DNA
aaccaataaaacctactcctcccttaa

SEQ ID NO: 11 - DNA, 30 bp, Artificial sequence
  Beta-actin probe (FAM--TAMRA, or FAM--Dabcyl) for chemically treated genomic DNA
accaccacccaacacacaataacaaacaca

SEQ ID NO: 12 - DNA, 21 bp, Artificial sequence
  PITX2 multiplex PCR forward primer for chemically treated genomic DNA
gtaggggagggaagtagatgt

SEQ ID NO: 13 - DNA, 23 bp, Artificial sequence
  PITX2 multiplex PCR reverse primer for chemically treated genomic DNA
tcctcaactctacaaacctaaaa

SEQ ID NO: 14 - DNA, 16 bp, Artificial sequence
  PITX2 detection oligonucleotide for chemically treated genomic DNA
agtcgggagagcgaaa

SEQ ID NO: 15 - DNA, 16 bp, Artificial sequence
  PITX2 detection oligonucleotide 2 for chemically treated genomic DNA
agttgggagagtgaaa

SEQ ID NO: 16 - DNA, 25 bp, Artificial sequence
  Beta-actin control primer 1 for chemically treated genomic DNA
tggtgatggaggaggtttagtaagt

SEQ ID NO: 17 - DNA, 27 bp, Artificial sequence
  Beta-actin control primer 2 for chemically treated genomic DNA
aaccaataaaacctactcctcccttaa

SEQ ID NO: 18 - DNA, 29 bp, Artificial sequence
  Beta-actin control probe (FAM--TAMRA) for chemically treated genomic DNA
ccaccacccaacacacaataacaaacaca

SEQ ID NO: 19 - DNA, 22 bp, Artificial sequence
  PITX forward primer for chemically treated genomic DNA
gtaggggagggaagtagatgtt

SEQ ID NO: 20 - DNA, 24 bp, Artificial sequence
  PITX reverse primer for chemically treated genomic DNA
ttctaatcctcctttccacaataa

SEQ ID NO: 21 - DNA, 20 bp, Artificial sequence
  PITX 2cg1 probe (FAM--Darquencher, or FAM--TAMRA) for chemically treated genomic DNA
agtcggagtcgggagagcga

SEQ ID NO: 22 - DNA, 20 bp, Artificial sequence
  PITX 2cp1 alternate probe (YAKIMA YELLOW-- Darquencher) for chemically treated genomic DNA
agtcggagtcgggagagcga